(19) United States
(12) Patent Application Publication
(10) Pub. No.: US 2017/0107554 A1
(43) Pub. Date: Apr. 20, 2017

(54) METHOD FOR PRODUCING ASTAXANTHIN

(71) Applicants: NIPPON SUISAN KAISHA, LTD., Tokyo (JP); BIOGENIC CO., LTD., Tokyo (JP)

(72) Inventors: Hitoshi IZUMIDA, Tokyo (JP); Eiji OHASHI, Tokyo (JP); Toru NUMASAWA, Tokyo (JP)

(21) Appl. No.: 15/128,907

(22) PCT Filed: Feb. 5, 2015
(86) PCT No.: PCT/JP2015/053220

§ 371 Date: Sep. 23, 2016

(30) Foreign Application Priority Data
Apr. 3, 2014 (JP) 2014-077246

(51) Int. Cl. C12P 23/00; C12N 1/12

(52) U.S. Cl. CPC C12P 23/00; C12N 1/12

(57) ABSTRACT

A method for increasing efficiency of a method for producing astaxanthin by culturing a microalga. A method for producing astaxanthin in which astaxanthin is produced in an algal body by culturing a microalga, wherein photoirradiation is performed using both a blue LED of peak wavelength from 420 to 500 nm and a red LED of peak wavelength from 620 to 690 nm, at least during an astaxanthin-producing culturing phase of a culturing period. The ratio of the blue LED of peak wavelength from 420 to 500 nm and the red LED of peak wavelength from 620 to 690 nm is preferably from 1:19 to 19:1 by photon flux density, and the photon flux densities are each preferably not less than 20 μmol/m²/s.

### TECHNICAL FIELD

The present invention relates to an efficient method for producing astaxanthin. More particularly, the present invention relates to photoirradiation when culturing microalgae that produce astaxanthin.

### BACKGROUND

Astaxanthin is a type of carotenoid having a red-orange color, and is the pigment contained in large quantity primarily in marine organisms such as crustaceans like shrimp and crab, salmon, salmon roe, sea bream, algae, and the like. Astaxanthin is known to have a powerful antioxidant action, and is used in food coloring, cosmetics, health foods, medicines, and the like.

Astaxanthin is produced by chemical synthesis or by culturing bacteria, yeasts, microalgae, and the like. When culturing bacteria or yeasts, not more than 2% by weight of astaxanthin per dry weight of bacteria or yeast is obtained, whereas by culturing microalgae of the Haematococcus genus (referred to as “Haematococcus algae” hereinafter), a high content of not less than 2% by weight is obtained, and due to its safety, it is produced worldwide. To produce astaxanthin using microalgae that photosynthesize Haematococcus algae and the like, photoirradiation suitable for their growth is required.

Astaxanthin is produced by microalgae such as Haematococcus algae, Chlorella, and Scenedesmus, for example. In particular, due to stress caused by changes in the external environment, Haematococcus algae are encysted and accumulate astaxanthin in the algal body. To accumulate astaxanthin, irradiation by sunlight or artificial light is required. Artificial light sources that are used include fluorescent lamps, light emitting diodes (LEDs), and the like.

If cultured using only sunlight, stable and efficient production is difficult because it is affected by fluctuations in air temperature and fluctuations in duration of daylight. For this reason, culturing using the artificial light source of a fluorescent lamp has been attempted since long ago.

Patent Document 1 describes that astaxanthin content of 2% by weight per weight of dry algal body was obtained in a working example in which Haematococcus algae were cultured under irradiation by artificial light of illuminance 40,000 lux.

Patent Document 2 describes a working example in which astaxanthin was produced highly efficiently with astaxanthin content of 6.8% by weight per weight of dry algal body and astaxanthin production quantity per volume of culture solution of 250 mg/L in 21 days by culturing Haematococcus algae under extremely high light intensity conditions with a photosynthesis-effective photon flux of 25,000 μmol-photon/m³/s, but the astaxanthin production quantity did not reach not less than 300 mg/L. To obtain such an extremely high photosynthesis-effective photon flux input quantity using fluorescent lamps, a large amount of electrical power is required, and furthermore, the amount of power required for the air conditioning system to control the heat generated by the fluorescent lamps is also large.

LEDs are known as a light source that is low power and produces little heat, and the production of astaxanthin using LEDs instead of fluorescent lamps has been examined.

In Patent Document 3, as a result of examining astaxanthin production from Haematococcus algae using LEDs of various wavelengths, high astaxanthin productivity was obtained by irradiation by only blue LEDs having a wavelength not greater than 540 nm. Particularly when a blue LED having a center wavelength of 470 nm was used, approximately twice the amount of astaxanthin was produced compared to fluorescent lamps of the same photon flux density. However, the astaxanthin concentration per volume of culture solution at that time was a low 25 mg/L after 12 days of culturing, and did not reach a culture concentration practical for industrial production.

Patent Document 4 describes that an increase in the cell count of Haematococcus algae was enhanced by irradiating Haematococcus alga colonies on an agar plate alternately with blue LEDs and red LEDs. However, it does not mention the astaxanthin production stage after encystation, nor does it describe whether or not astaxanthin was produced. Since it is known that encystation of Haematococcus algae occurs due to stress after maturation and that a large quantity of astaxanthin is accumulated, it is impossible to judge whether or not astaxanthin production increases in Patent Document 4.

### PRIOR ART DOCUMENTS

### Patent Documents

Patent Document 1: Japanese Unexamined Patent Application Publication No. H3-83577A

Patent Document 2: Japanese Unexamined Patent Application Publication No. 2007-97584A

Patent Document 3: Japanese Unexamined Patent Application Publication No. 2004-147641A

Patent Document 4: WO2013/021675

### SUMMARY OF THE INVENTION

### Problems to be Solved by the Invention

There has been demand for a culturing method using LEDs of low power and low heat generation to produce a higher content of astaxanthin than when microalgae are cultured using only blue LEDs having a wavelength not greater than 540 nm, and to produce astaxanthin in a astaxanthin concentration not less than 100 mg/L.

### Means for Solving the Problems

An object of the present invention is to produce astaxanthin more efficiently than with fluorescent lamps, using LEDs that can save power and suppress a temperature increase in the portion that transmits light.

As a result of diligent research to solve the above object, the present inventors discovered that astaxanthin is efficiently produced by culturing microalgae under simultaneous irradiation by both a blue LED of peak wavelength from 420 to 500 nm and a red LED of peak wavelength from 620 to 690 nm.

The gist of the present invention comprises methods (1) to (6) below for producing astaxanthin.

(1) A method for producing astaxanthin in which astaxanthin is produced in an algal body by culturing a microalga, wherein photoirradiation is performed using both a blue LED of peak wavelength from 420 to 500 nm and a red LED of peak wavelength from 620 to 690 nm, at least during an astaxanthin-producing culturing phase of a culturing period.

(2) The method for producing astaxanthin according to (1), wherein a ratio of the blue LED of peak wavelength from 420 to 500 nm and the red LED of peak wavelength from 620 to 690 nm is from 1:19 to 19:1 by photon flux density.

(3) The method for producing astaxanthin according to (1) or (2), wherein photon flux densities of the blue LED of peak wavelength from 420 to 500 nm and the red LED of peak wavelength from 620 to 690 nm are each not less than 20 μmol/m²/s.

(4) The method for producing astaxanthin according to any of (1) to (3), wherein the microalga is a Haematococcus genus alga.

(5) The method for producing astaxanthin according to any of (1) to (4), wherein an astaxanthin production quantity per volume of culture solution is not less than 100 mg/L.

(6) The method for producing astaxanthin according to (5), wherein the astaxanthin production quantity per volume of culture solution is not less than 300 mg/L.

(7) A culture solution of a microalga, wherein an astaxanthin content is not less than 300 mg/L.

(8) A cultured algal body of a microalga, wherein an astaxanthin content is not less than 7.0% by weight (in a dry algal body).

### Effect of the Invention

The present invention enables efficient production of astaxanthin without greatly modifying conventional astaxanthin production methods or equipment.

### BRIEF DESCRIPTION OF THE DRAWINGS

FIG. 1 is a drawing illustrating spectra of a blue LED and a red LED used in Working Example 1.

FIG. 2 is a drawing illustrating dry alga body weight per volume of culture solution in Working Example 2.

FIG. 3 is a drawing illustrating astaxanthin content per dry algal body in Working Example 2.

FIG. 4 is a drawing illustrating astaxanthin production quantity per volume of culture solution in Working Example 2.

### MODE FOR CARRYING OUT THE INVENTION

The present invention relates to a method for producing astaxanthin using microalgae, containing a step of irradiating microalgae with a blue LED of peak wavelength from 420 to 500 nm and a red LED of peak wavelength from 620 to 690 nm.

In the present invention, a microalga capable of producing astaxanthin can be used. The microalga stated here is limited to those that perform photosynthesis. Known microalgae include cyanobacteria, Rhodophyta, Phaeophyceae, Chlorophyceae, Bacillariophyceae, Eustigmatophyceae, and the like, but the microalga of the present invention is limited to microalgae capable of producing astaxanthin. As microalgae that produce astaxanthin, microalgae belonging to the Haematococcus genus (Haematococcus algae) are generally used.

As Haematococcus algae, Haematococcus lacustris, H. pluvialis, H. capensis, H. droebakensi, H. zimbabwiensis, and the like may be used. Among them, Haematococcus lacustris and Haematococcus pluvialis are preferably used.

Microalgae other than those of the Haematococcus genus that produce astaxanthin may also be used. Examples include microalgae of Chlorella zofingiensis, which is a Chlorella genus alga, and Monoraphidium sp. alga, as well as Vischeria helvetica, Coelastrella, Scenedesmus, Chlamydomonas nivalis, Protosiphon botryoides, Neochloris wimmeri, and the like.

The culture medium used in culturing of the microalga is not particularly limited, but is preferably an autotrophic medium not containing a carbon source to prevent contamination of the medium. An autotrophic medium containing nitrogen, trace amounts of inorganic metal salts, vitamins, and the like required for growth is generally used. For example, media such as VT medium, C medium, MC medium, MBM medium, MDM medium, and the like (refer to Alga Research Methods, Nishizawa, K. and Chihara, M., Kyoritsu Shuppan (1979)), BG-11 medium, and modified media thereof are used.

Furthermore, when culturing microalgae in a medium, it is preferable to ventilate with air containing carbon dioxide. The microalgae may be cultured while ventilating with air not containing carbon dioxide, but since that retards the growth of the microalgae, they are cultured while ventilating with air containing from 0.1 to 5% carbon dioxide, and more preferably from 0.5 to 3% carbon dioxide. It is possible to culture the microalgae without ventilation, but for good development, the air flow rate is from 0.01 to 3.0 vvm and preferably from 0.015 to 1 vvm, and the pH is from 5 to 10 and preferably from 6 to 9.

As for culturing temperature, when using Haematococcus lacustris and Haematococcus pluvialis, the culturing temperature is, for example, from 10 to 45° C. and preferably from 18 to 38° C. The pH of the culture medium is adjusted in the range from 5.0 to 9.5 and preferably in the range from 6.0 to 9.0.

Photoirradiation of the microalgae for astaxanthin production is performed using both a blue LED of peak wavelength from 420 to 500 nm and a red LED of peak wavelength from 620 to 690 nm. The microalgae needs to be irradiated by both the blue LED and the red LED during all or a certain portion of the microalga culturing period. In particular, it is important to irradiate the microalgae using both the blue LED and the red LED during the astaxanthin-producing culturing phase (cyst cell phase). If irradiated by both the blue LED and the red LED, astaxanthin can be produced with the greatest efficiency by simultaneous irradiation, but astaxanthin can also be produced efficiently by alternately irradiating by the blue LED and the red LED within 24 hours. Alternatively, an irradiation method wherein the blue LED and the red LED blink alternately may be used.

As the light source in the photoirradiation step, an LED, an incandescent bulb, a fluorescent lamp, and the like may be used, but light sources other than LEDs have poor efficiency because the wavelength spectrum of the light source spans a range, and therefore unnecessary light needs to be cut. If an LED is used, astaxanthin can be efficiently produced with low radiation energy because radiation of light with a narrow wavelength range is possible without requiring a special means to cut some of the light. An organic EL light source may also be used as the LED.

It is preferable to use a plurality of LED chips so that efficient irradiation is performed. If a plurality of light sources are used, it is preferable to dispose the light sources at equal intervals to enable as uniform light radiation as possible. Furthermore, a plurality of chips of blue LEDs and red LEDs may be made into independent panels to radiate light, or irradiation may be performed using a panel embedded with a plurality of chips of blue LEDs and red LEDs in a certain proportion.

The irradiated wavelength of the blue LED is a peak wavelength from 420 to 500 nm and preferably from 430 to 490 nm, and the wavelength of the red LED is from 620 to 690 nm and preferably from 630 to 680 nm.

Blue LEDs and red LEDs that emit light of not less than two different peak wavelengths may also be used. For example, irradiation is possible using blue LEDs of peak wavelengths 430 nm and 470 nm and red LEDs of peak wavelengths 630 nm and 660 nm.

The blue LEDs and red LEDs preferably emit light having a narrow width of wavelength. This is because more efficient astaxanthin production is possible by selective irradiation by selecting only the range of wavelength suitable for astaxanthin production.

The ratio of blue LEDs of peak wavelength from 420 to 500 nm and red LEDs of peak wavelength from 620 to 690 nm that radiate simultaneously during microalga culturing is not limited provided that they radiate simultaneously, but the ratio is from 1:19 to 19:1 and preferably from 1:5 to 5:1 by photon flux density. A ratio from 1:2.5 to 5:1 is more preferable, and from 1:2 to 4:1 is particularly preferable.

The light radiation method is also not particularly limited, and may be, for example, continuous radiation or intermittent radiation at a set interval. Here, “intermittent radiation” includes radiation with pulsed light. If light is intermittently irradiated, power consumption can be reduced.

Haematococcus algae such as Haematococcus lacustris and Haematococcus pluvialis take the form of green vegetative cells having motility and robust cell growth, and also take the form of cyst cells encysted due to stress from extreme changes in environmental conditions such as temperature, intense light, salt, moisture content, nutrients, and the like. When encysted, they accumulate astaxanthin in the algal body and turn red.

Photoirradiation using blue LEDs of peak wavelength from 420 to 500 nm and red LEDs of peak wavelength from 620 to 690 nm may be used both when the alga is in the form of vegetative cells and in the form of cyst cells. Vegetative cells produce a slight amount of astaxanthin, but since their production rate is slow, they are effective for obtaining somewhat good cell division and growth. In the cyst cell phase, astaxanthin can be efficiently produced because the astaxanthin production rate is fast and it accumulates in a high concentration.

During the initial phase of culturing of Haematococcus algae, they are motile, there are numerous vegetative cells, and cell density is low, and therefore they can be grown well with photon flux density of not greater than 20 μmol/m2/s. When cultured in the form of vegetative cells, they grow well even if a light source other than an LED is used. They can also be cultured using only either blue LEDs of peak wavelength from 420 to 500 nm or red LEDs of peak wavelength from 620 to 690 nm.

The photon flux density when culturing after encystation of Haematococcus algae by applying stress such as temperature, intense light, or salt is not particularly limited, but if a culturing apparatus having a light transmission width (diameter, thickness) of, for example, not greater than 70 mm is used, astaxanthin can be efficiently produced by irradiation by blue LEDs of peak wavelength from 420 to 500 nm and red LEDs of peak wavelength from 620 to 690 nm each having a photon flux density of not less than 20 μmol/m²/s, preferably not less than 50 μmol/m²/s, and more preferably not less than 100 μmol/m²/s or not less than 150 μmol/m²/s. If a culturing apparatus with a light transmission width greater than that is used, it may be even larger. That is, when culturing Haematococcus algae in the form of cyst cells, astaxanthin can be produced efficiently by irradiation by both blue LEDs and red LEDs There is no particular upper limit of photon flux density, but from the perspective of balancing energy costs and effect, not greater than 3000 μmol/m²/s is preferred, and not greater than 1000 μmol/m²/s is particularly preferred.

Through the above culturing, it is possible to obtain a culture solution containing astaxanthin (as a free form) in a concentration of not less than 100 mg/L of culture solution, preferably not less than 300 mg/L, and more preferably not less than 400 mg/L. It is also possible to obtain a cultured algal body of microalgae having an astaxanthin content of not less than 7.0% by weight (in the dry algal body).

The method for recovering astaxanthin from the culture solution is not particularly limited. For example, dry microalgae may be obtained by separating the microalga culture solution containing astaxanthin by solid-liquid separation means such as filtration and centrifugation to collect microalga cells, and then drying them (natural drying, drum drying, hot air drying, spray drying, freeze drying, and the like). The obtained dried microalga product contains astaxanthin (as a free substance) in a concentration from 1 to 10% by mass. The concentration is preferably from 4 to 10% by mass.

A component containing astaxanthin may be obtained by crushing the wet algal body or the above dried product containing astaxanthin, and extracting and recovering astaxanthin. The methods of extraction and recovery of astaxanthin are not particularly limited, but methods commonly used by persons skilled in the art may be used. For example, astaxanthin is extracted after the dried microalga product is mechanically crushed. Examples of the extraction method include chemical extraction using an organic solvent such as chloroform, hexane, acetone, methanol, ethanol, and edible oils and fats, or physical extraction by expression of dried Chlorophyceae, and the like. Alternatively, it may be extracted or recovered using supercritical extraction. The extraction solvent is distilled out to obtain oil containing astaxanthin.

Methods of LED irradiation of the culture solution include external irradiation in which a culture solution contained in a reactor is irradiated from the outside, and internal irradiation in which LEDs are put into a culture solution contained in a reactor, but either may be used without particular limitation. Note that the value used as photon flux density in the case of external irradiation is that measured on the exterior surface of the container, and in the case of internal irradiation, it is the value at the container surface in contact with the culture solution. Both external irradiation and internal irradiation can be used together.

The microalga culturing apparatus for astaxanthin production is not particularly limited provided that carbon dioxide can be supplied and the culture solution can be photoirradiated using both a blue LED of peak wavelength from 420 to 500 nm and a red LED of peak wavelength from 620 to 690 nm. For example, if on a small scale, a flat culture bottle from 10 to 50 mm thick or a glass tube from approximately 20 to 70 mm in diameter is preferably used. If on a large scale, a culture vessel constructed from a plastic bag or a tube or transparent plate made of glass, plastic, or the like, equipped with a light and a stirrer as necessary, is used. When culturing on a large scale, the light transmission width (diameter, thickness) is preferably not greater than 400 mm, and more preferably not greater than 70 mm. Examples of such a culture vessel include a flat panel culture vessel, tube culture vessel, air dome culture vessel, hollow cylinder culture vessel, internally illuminated tank culture vessel, and the like. In any case, a tightly sealing container is preferably used. For example, a type in which a tube is coiled around LEDs as disclosed in Japanese Unexamined Patent Application Publication No. 2012-29578A, or a hybrid type of reactor as disclosed in Japanese Unexamined Patent Application Publication No. 2014-39491A may be used.

Types of culturing of astaxanthin include placing a vessel outdoors and using sunlight, and placing a vessel indoors and using artificial light. The method that uses sunlight can produce astaxanthin inexpensively because there are no energy costs, but if the equipment is crude, quality may decrease due to impurities or contaminants. The present invention may be used with either type. Even when using natural light, the effect of the present invention can be obtained by using both a blue LED of peak wavelength from 420 to 500 nm and a red LED of peak wavelength from 620 to 690 nm at least during the astaxanthin-producing culturing phase of the culturing period.

When culturing using only artificial light, both a blue LED of peak wavelength from 420 to 500 nm and a red LED of peak wavelength from 620 to 690 nm are used at least during the astaxanthin-producing culturing phase. Other light sources such as a fluorescent lamp may be used during the growth culturing phase, but both blue light and red light may also be used, similar to the astaxanthin-producing culturing phase.

The ratio of photon flux density of blue light and red light is from 1:19 to 19:1 and preferably from 1:5 to 5:1. A ratio from 1:2.5 to 5:1 is more preferable, and from 1:2 to 4:1 is particularly preferable.

The present invention is described in detail below using working examples, but the present invention is not limited by these examples.

In the present invention, astaxanthin quantity was measured by the following method.

Astaxanthin Measurement by HPLC Using Luna 3 Um Silica Column

A certain quantity of sample is collected, acetone is added, and the resulting product is crushed. After centrifugal separation, the supernatant is recovered. 0.05 M Tris-HCl buffer and cholesterol esterase solution are added to the supernatant and reacted for 45 minutes at 37° C., and astaxanthin is freed. The astaxanthin is extracted with petroleum ether, the solvent is distilled out, and the resulting product is dried. This is dissolved in hexane:acetone=82:18, and used as a sample solution for HPLC. It is measured under the HPLC analysis conditions given below. Because astaxanthin has geometric isomers, astaxanthin content is analyzed based on their peak area.

HPLC Analysis Conditions

Column: Luna 3 μm Silica (2) 100A 150*4.6 mm (Phenomenex Inc.)

Mobile phase solvent: hexane:acetone=82:18 (v/v)

Device start-up method: A-JUNSOU

A-JUNSOU method settings

Injected sample quantity: 20 μL

Mobile phase flow rate: 1.2 mL/min

Column temperature: 30° C.

DAD: 455 nm, 467 nm, 475 nm

Measurement time: 13 min

### Working Example 1

Culturing of Haematococcus (Growth Culturing)

15 mL of culture solution, containing vegetative cells of Haematococcus lacustris strain NIES144 (preserved at the National Institute for Environmental Studies Microbial Culture Collection facility) in a concentration of 500,000 cells/mL, and 750 mL of BG11 modified A medium (Table 1) were poured into each of four transparent glass culture vessels 50 mm in inner diameter and 500 mm high. The cells were cultured at 25° C. while stirring and ventilating with air containing 1% carbon dioxide, under continuous irradiation by a fluorescent lamp so as to result in a photon flux density of 50 μmol/m²/s. As a result, growth of 450,000 vegetative cells/mL was seen in each culture vessel on day 5 of culturing.

Culturing of Haematococcus Using Various Light Sources (Astaxanthin-Producing Culturing)

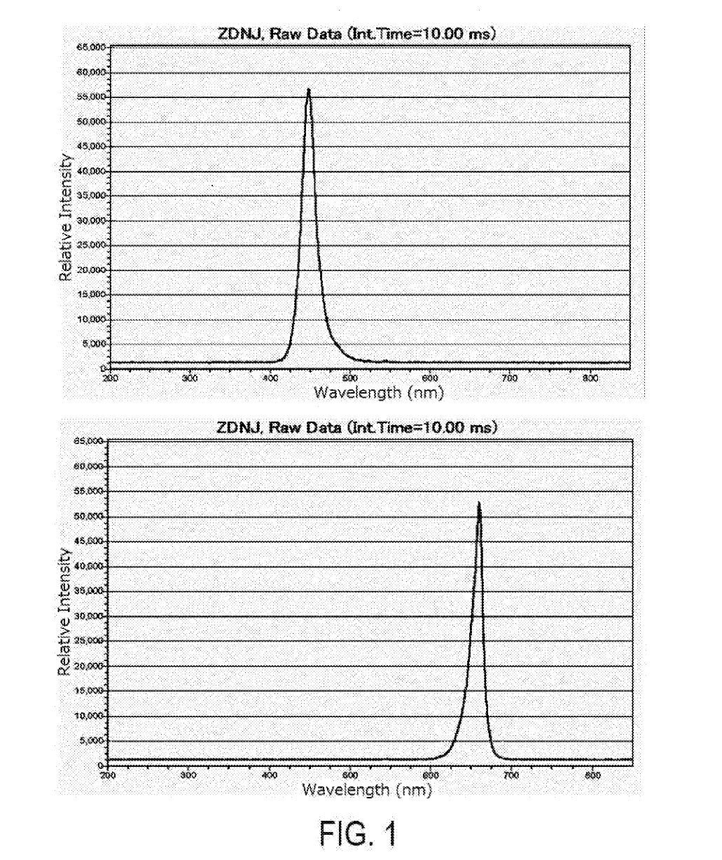

Then, after sodium chloride was added to each culture solution so as to result in a concentration of 2 g/L, light was radiated from seven types of light source so as to result in a photon flux density of 300 μmol/m²/s each, and astaxanthin was produced at 27° C. while stirring and ventilating with air containing 1% carbon dioxide. The light sources at this time were a fluorescent lamp, a blue LED of wavelength 450 nm that radiated alone, a red LED of wavelength 660 nm that radiated alone, and a blue LED of wavelength 450 nm and a red LED of wavelength 660 nm that radiated simultaneously and continuously (at four ratios of blue light and red light, namely 1:2, 1:1, 2:1, 4:1). The spectra of the blue LED and red LED used in this experiment are shown in FIG. 1. After culturing for 14 days, a dry algal body was obtained by filtration. The dry algal body was weighed, and the dry algal body weight per volume of culture solution was determined. Furthermore, the astaxanthin content in the dry algal body and the astaxanthin production quantity per volume of culture solution were determined by reversed-phase HPLC.

TABLE 1

| Ingredient | | Micro element solution | | Vitamin solution | |
|---|---|---|---|---|---|
| NaNO₃ | 480 mg | ZnSO₄•7H₂O | 22.2 mg | Thiamine | 100 mg |
| CaCl₂•2H₂O | 21.6 mg | MnCl₂•4H₂O | 181 mg | B₁₂ | 1.25 mg |
| Na₂CO₃ | 12 mg | CuSO₄•5H₂O | 7.9 mg | Biotin | 12.5 mg |
| MgSO₄•7H₂O | 60 mg | Co(NO₃)₂•6H₂O | 49 mg | Distilled water | 100 ml |
| K₂HPO₄ | 30.6 mg | H₃BO₃ | 286 mg | | |
| Citric acid | 5.4 mg | Na₂MoO₄•2H₂O | 39 mg | | |
| Ammonium iron (iii) citrate | 3.6 mg | Na₂EDTA | 100 mg | | |
| Micro element solution | 0.3 ml | Distilled water | 50 ml | | |
| Vitamin solution | 0.12 ml | | | | |
| Distilled water | 1000 ml | | | | |

The results are shown in Table 2.

When photoirradiation was performed only with a blue LED, the dry algal body weight was 2.4 g/L, which was lower than the case of a fluorescent lamp, but the astaxanthin content was 3.9% by weight and the astaxanthin production quantity per volume of culture solution was 94 mg/L, which were higher. When photoirradiation was performed only with a red LED, the dry algal body weight was 3.3 g/L, which was higher than the case of a fluorescent lamp, but the astaxanthin content was 1.3% by weight and the astaxanthin production quantity per volume of culture solution was 43 mg/L, which were lower.

When the blue LED and red LED radiated simultaneously, the astaxanthin production quantities at all of the photon flux density ratios of 1:2, 1:1, 2:1, and 4:1 of blue and red light were higher than the case of a fluorescent lamp, at 131 mg/L, 162 mg/L, 155 mg/L, and 156 mg/L, respectively. When blue light and red light were both used, the astaxanthin production quantity was greatly improved in all cases compared to the cases of a fluorescent lamp, blue light alone, and red light alone. It was found that a ratio of blue light and red light from 1:2 to 4:1 is preferable. In particular, as a result of simultaneous continuous irradiation at a ratio of 1:1, the dry algal body weight was 3.3 g/L, similar to the case of a fluorescent lamp, but the astaxanthin content was 4.9% by weight, and the astaxanthin concentration per volume of the culture solution was 162 mg/L, which was twice the value of the case of a fluorescent lamp and 1.7 times the value of the case of the blue LED.

From the above results, it was ascertained that by simultaneously radiating a blue LED and a red LED during the astaxanthin-producing culturing phase of the culturing period, the astaxanthin content of the algal body is increased, and as a result, the astaxanthin production quantity per volume of culture solution can be increased.

TABLE 2

| | | Dry algal | Astaxanthin | Astaxanthin |
|---|---|---|---|---|
| | Light | body | content (in | production |
| | intensity | weight | algal body) | quantity |
| Light source | μmol/m²/s | g/L | wt. % | mg/L |
| Fluorescent lamp | 300 | 2.9 | 2.8 | 81 |
| Blue LED | 300 | 2.4 | 3.9 | 94 |
| Red LED | 300 | 3.3 | 1.3 | 43 |
| Blue LED:Red LED | 300 | 3.2 | 4.1 | 131 |
| 1:2 | | | | |
| Blue LED:Red LED | 300 | 3.3 | 4.9 | 162 |
| 1:1 | | | | |
| Blue LED:Red LED | 300 | 3.1 | 5.0 | 155 |
| 2:1 | | | | |
| Blue LED:Red LED | 300 | 3.0 | 5.2 | 156 |
| 4:1 | | | | |

### Working Example 2

Culturing of Haematococcus (Growth Culturing)

15 mL of culture solution, containing vegetative cells of Haematococcus lacustris strain NIES144 in a concentration of 500,000 cells/mL, and 750 mL of BG11 modified B medium (Table 3) were poured into a transparent glass culture vessel 50 mm in inner diameter and 500 mm high. The cells were cultured at 25° C. while stirring and ventilating with air containing 1% carbon dioxide, under simultaneous continuous irradiation by a blue LED of wavelength 450 nm (photon flux density 50 μmol/m²/s) and a red LED of wavelength 660 nm (photon flux density LED 30 μmol/m²/s). As a result, growth of 360,000 vegetative cells/mL was seen on day 4 of culturing.

Culturing of Haematococcus (Astaxanthin-Producing Culturing)

Then, after sodium chloride was added to the culture solution so as to result in a concentration of 2 g/L, astaxanthin was produced at 28° C. while stirring and ventilating with air containing 1% carbon dioxide under simultaneous continuous irradiation by a blue LED of wavelength 450 nm (photon flux density LED 300 μmol/m²/s) and a red LED of wavelength 660 nm (photon flux density 250 μmol/m²/s). Changes over time were observed for 21 days of culturing. A dry algal body was obtained by filtration and then weighed, and the dry algal body weight per volume of culture solution was determined. The astaxanthin content and the astaxanthin concentration per volume of culture solution were determined by reversed-phase HPLC.

TABLE 3

| Ingredient | | Micro element solution | | Vitamin solution | |
|---|---|---|---|---|---|
| NaNO₃ | 720 mg | ZnSO₄•7H₂O | 22.2 mg | Thiamine | 100 mg |
| CaCl₂•2H₂O | 32.4 mg | MnCl₂•4H₂O | 181 mg | B₁₂ | 1.25 mg |
| Na₂CO₃ | 18 mg | CuSO₄•5H₂O | 7.9 mg | Biotin | 12.5 mg |
| MgSO₄•7H₂O | 90 mg | Co(NO₃)₂•6H₂O | 49 mg | Distilled water | 100 ml |
| K₂HPO₄ | 45.9 mg | H₃BO₃ | 286 mg | | |
| Citric acid | 8.1 mg | Na₂MoO₄•2H₂O | 39 mg | | |
| Ammonium iron (iii) citrate | 5.4 mg | Na₂EDTA | 100 mg | | |
| Micro element solution | 0.45 mL | Distilled water | 50 ml | | |
| Vitamin solution | 0.18 mL | | | | |
| Distilled water | 1000 mL | | | | |

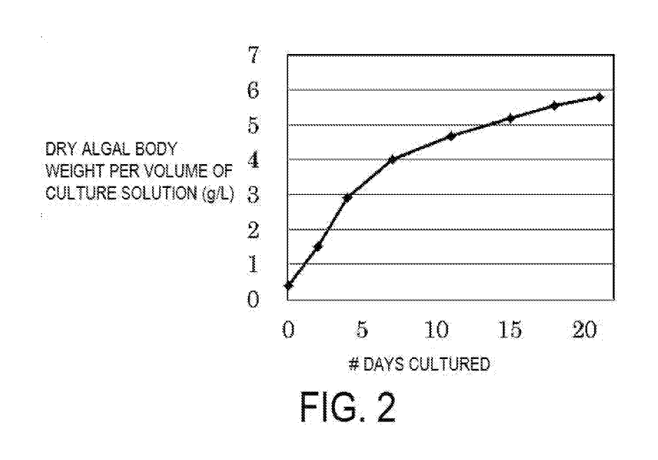

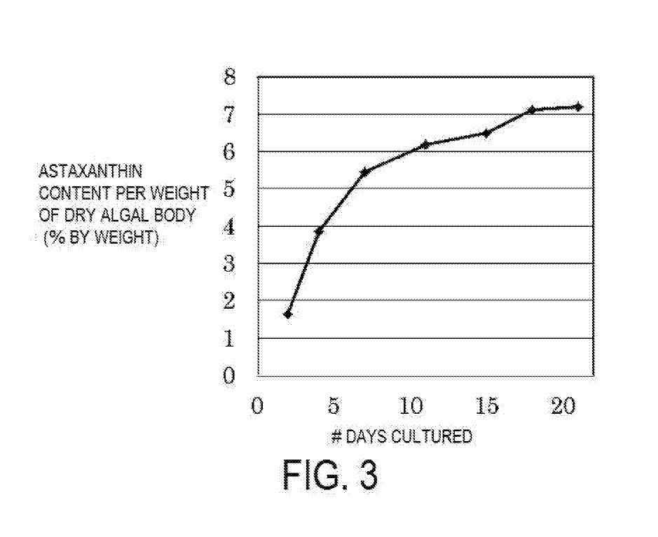

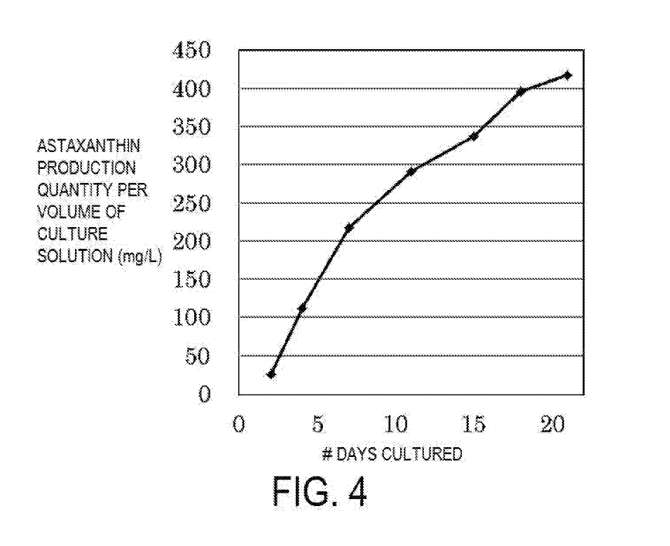

The results of dry algal body weight per volume of culture solution, astaxanthin content (% by weight), and astaxanthin production quantity per volume of culture solution (mg/L) versus the number of days of culturing after sodium chloride was added are shown in FIGS. 2 to 4. On day 21 of culturing, dry algal body weight was 5.8 g/L, astaxanthin content in the dry algal body was 7.2% by weight, and astaxanthin produced quantity was 418 mg/L.

### INDUSTRIAL APPLICABILITY

By the method of the present invention, the astaxanthin production quantity per volume of culture solution can be increased while using a small amount of energy.

1. A method for producing astaxanthin in which astaxanthin is produced in an algal body by culturing a microalga, wherein photoirradiation is performed using both a blue LED of peak wavelength from 420 to 500 nm and a red LED of peak wavelength from 620 to 690 nm, at least during an astaxanthin-producing culturing phase of a culturing period.

2. The method for producing astaxanthin according to claim 1, wherein a ratio of the blue LED of peak wavelength from 420 to 500 nm and the red LED of peak wavelength from 620 to 690 nm is from 1:19 to 19:1 by photon flux density.

3. The method for producing astaxanthin according to claim 1, wherein photon flux densities of the blue LED of peak wavelength from 420 to 500 nm and the red LED of peak wavelength from 620 to 690 nm are each not less than 20 μmol/m²/s.

4. The method for producing astaxanthin according to claim 1, wherein the microalga is a Haematococcus genus alga.

5. The method for producing astaxanthin according to claim 1, wherein an astaxanthin production quantity per volume of culture solution is not less than 100 mg/L.

6. The method for producing astaxanthin according to claim 5, wherein the astaxanthin production quantity per volume of culture solution is not less than 300 mg/L.

7. A culture solution of a microalga, wherein an astaxanthin content is not less than 300 mg/L.

8. A cultured algal body of a microalga, wherein an astaxanthin content is not less than 7.0% by weight (in a dry algal body).

9. The method for producing astaxanthin according to claim 2, wherein photon flux densities of the blue LED of peak wavelength from 420 to 500 nm and the red LED of peak wavelength from 620 to 690 nm are each not less than 20 μmol/m²/s.

10. The method for producing astaxanthin according to claim 2, wherein the microalga is a Haematococcus genus alga.

11. The method for producing astaxanthin according to claim 3, wherein the microalga is a Haematococcus genus alga.

12. The method for producing astaxanthin according to claim 2, wherein an astaxanthin production quantity per volume of culture solution is not less than 100 mg/L.

13. The method for producing astaxanthin according to claim 3, wherein an astaxanthin production quantity per volume of culture solution is not less than 100 mg/L.

14. The method for producing astaxanthin according to claim 4, wherein an astaxanthin production quantity per volume of culture solution is not less than 100 mg/L.
